(19) United States
(12) Patent Application Publication
(10) Pub. No.: US 2007/0248771 A1
(43) Pub. Date: Oct. 25, 2007

(54) Acenaphtho[1,2-b]quinoxaline sulfo-and carboxy-derivative, lyotropic liquid crystal system, optically anisotropic film and method thereof and laminated optical film

(75) Inventors: Alexey Nokel, Moscow (RU); Tatsuki Nagatsuka, Osaka (JP); Michael V. Paukshto, Foster City, CA (US)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: NITTO DENKO CORPORATION

(21) Appl. No.: 11/408,357

(22) Filed: Apr. 21, 2006

(51) Int. Cl. C09K 19/00; C07D 241/36

(52) U.S. Cl. 428/1.31; 544/342

(57) ABSTRACT

An acenaphtho[1,2-b]quinoxaline sulfo- and carboxy-derivative of the present invention is represented by a structure formula selected from the group consisting of structures I: 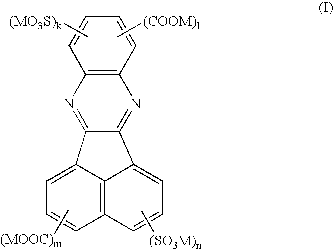 wherein: k, l are individually integers in the range of 0 to 4; m, n are individually integers in the range of 0 to 6; M is a counter ion. The acenaphtho[1,2-b]quinoxaline sulfo- and carboxy-derivative can form a lyotropic liquid crystal system and is useful for an optically anisotropic film.

### BACKGROUND OF THE INVENTION

1. Field of the Invention

The present invention relates generally to the fields of organic chemistry and optically anisotropic coatings. More specifically, the present invention is related to synthesizing heterocyclic sulfo- and carboxy-derivative compounds and manufacturing optically anisotropic coatings based on these compounds.

2. Description of the Prior Art

Modern technological progress requires development of optical elements based on new materials with specific, controllable optical properties and high environmental stability. In particular, the necessary element in modern visual display systems is an optically anisotropic film that is optimized for the optical characteristics of an individual display module.

Various searched polymer materials like polycarbonate, cyclic polyolefin such as, ZEONEX, ZEONOR (registered trade mark) manufactured by ZEON CORPORATION, ARTON (registered trade mark) manufactured by JSR CORPORATION and others are known in the prior art [for example: E. L. Strebel, “1,3-Bis-(carboxy-phenylamino)-s-triazines” (1977), U.S. Pat. No. 4,031,092, (Minnesota Mining and Manufacturing Company); “Liquid Crystal Cell Which Can Have a Homeotropic Structure with Compensated Birefringence of Said Structure”, U.S. Pat. No. 4,701,028, 1987, (Commissariat a l'Energie Atomique); “Liquid Crystal Display Device Comprising a Retardation Compensation Layer Having a Maximum Principal Refractive Index in the Thickness Direction”, U.S. Pat. No. 5,124,824, 1992, (Mitsubishi Denki Kabushiki Kaisha)] for use in the production of A-plates and biaxial films. However, most of them require cross-lamination and cannot be used in roll-to-roll process forming an optical stack with conventional sheet polarizers.

Competitive technology of reactive LC coatings (for example, Merck—LG Chemical JV) requires substrate surface alignment (rubbing), further UV stabilization, and it is not free from point defects causing depolarization.

Therefore new transparent lyotropic liquid crystal (LLC) materials are very promising for the manufacture of optically anisotropic films with desirable optical and working characteristics. Films based on these materials are formed by wet-coating roll-to-roll process.

### SUMMARY OF THE INVENTION

It is an object of the present invention to provide a new carboxy-derivative useful for LLC materials and methods of their forming as well as some applications of the new optically anisotropic nano-films.

The present invention provides an acenaphtho[1,2-b]quinoxaline sulfo- and carboxy-derivative represented by a structure formula selected from the group consisting of structures I:

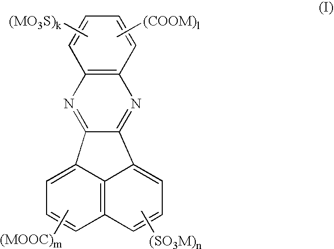

wherein: k, l are individually integers in the range of 0 to 4; m, n are individually integers in the range of 0 to 6; M is a counter ion.

The new compound provided by the present invention expands the assortment of compounds that are either not absorbing or only weakly absorbing in the visible spectral region and that are capable of forming a lyotropic liquid crystal (LLC) phase.

In the above acenaphtho[1,2-b]quinoxaline sulfo- and carboxy-derivative, said derivative is preferably capable of forming a stable lyotropic liquid crystal system.

In the above acenaphtho[1,2-b]quinoxaline sulfo- and carboxy-derivative, said derivative is preferably capable of forming optically isotropic or anisotropic film.

The present invention also provides a lyotropic liquid crystal system comprising at least one acenaphtho[1,2-b]quinoxaline sulfo- and carboxy-derivative represented by a structure formula selected from the group consisting of above structures I.

The above lyotropic liquid crystal system, preferably, further comprises a mixture of water and an organic solvent miscible with water.

The above lyotropic liquid crystal system, preferably, further comprises up to approximately 30% by mass of surfactants.

The above lyotropic liquid crystal system, preferably, further comprises up to approximately 30% by mass of plasticizers.

The above lyotropic liquid crystal system, preferably, further comprises at least one water-soluble organic compound capable of forming a common lyotropic liquid crystal system with at least one acenaphtho[1,2-b]quinoxaline sulfo- and carboxy-derivative of structure of I.

The present invention also provides an optically anisotropic film comprising at least one acenaphtho[1,2-b]quinoxaline sulfo- and carboxy-derivative represented by a structure formula selected from the group consisting of above structures I.

The present invention also provides a method of manufacturing an optically anisotropic film, comprising the step of:

depositing a lyotropic liquid crystal system onto a substrate; applying an orienting force; and drying. The lyotropic liquid crystal system comprises at least one acenaphtho[1,2-b]quinoxaline sulfo- and carboxy-derivative represented by a structure formula selected from the group consisting of structures I:

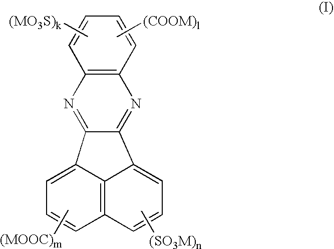

wherein: k, l are individually integers in the range of 0 to 4; m, n are individually integers in the range of 0 to 6; M is a counter ion.

In the above method of manufacturing the optically anisotropic film, said film is preferably formed between at least two substrates with oriented or rubbed surface from the above lyotropic liquid crystal system.

The above optically anisotropic film is preferably at least partially crystalline.

In the above optically anisotropic film, the interplane spacing in a crystal is preferably in the range of approximately 3.1 Å to 3.7 Å along one of the optical axes.

The above optically anisotropic film is useful for a birefringent film.

The above optically anisotropic film is useful for a negative A-plate.

The above optically anisotropic film is useful for a negative C-plate.

The above optically anisotropic film is useful for a polarizing plate.

The present invention also provides a laminated optical film in which at least one optically anisotropic film is laminated with an optical multilayer film having at least one polarizer, wherein

said optically anisotropic film comprises at least one acenaphtho[1,2-b]quinoxaline sulfo- and carboxy-derivative represented by a structure formula selected from the group consisting of above structures I.

In the above laminated optical film, said optically anisotropic film can be used as a negative A-plate.

In the above laminated optical film, said optically anisotropic film can be used as a negative C-plate.

The present invention also provides a laminated optical film in which at least one optically anisotropic film is laminated with an optical multilayer film having at least one retarder, wherein said optically anisotropic film comprises at least one acenaphtho[1,2-b]quinoxaline sulfo- and carboxy-derivative represented by a structure formula selected from the group consisting of above structures I.

### DETAILED DESCRIPTION OF THE INVENTION

The present invention is directed to synthesis of compounds, that are either not absorbing or only weakly absorbing in the visible spectral region and that are capable of forming a LLC phase. The water-soluble compounds of the present invention—acenaphtho[1,2-b]quinoxaline sulfo- and carboxy- derivatives, according to the invention, are represented by a structure formula selected from the group consisting of structures I:

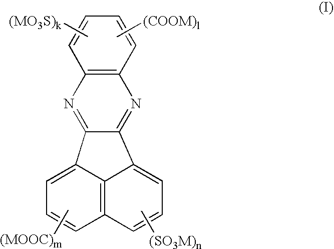

wherein: k, l are individually integers in the range of 0 to 4; m, n are individually integers in the range of 0 to 6; M is a counter ion.

Compounds corresponding to the structural formula comprise a whole class of structures differing by the number and positions of sulfonyl and carboxyl groups.

All of these compounds in individual form, as well as when blended with each other or with other known dichroic dyes and also in mixtures with some organic compounds that do not absorb in the visible region and, are capable of forming stable LLC phases.

Various cations, including for instance those selected from H⁺, NH₄⁺, K⁺, Li^{+,} Na⁺, Cs⁺, Ca²⁺, Sr²⁺, Mg²⁺, Ba²⁺, Co²⁺, Mn²⁺, Zn²⁺, Cu²⁺, Pb²⁺, Fe²⁺, Ni²⁺, Al³⁺, Ce³⁺ and others as well as mixtures of cations may be used as counterions in the structures described above.

Sulfo- and carboxy- derivatives of the general formula are formed at sulfonation of acenaphtho[1,2-b]quinoxaline carboxylic acids with sulfuric acid, chlorosulfonic acid or oleum at different concentrations in different temperature ranges as follows:

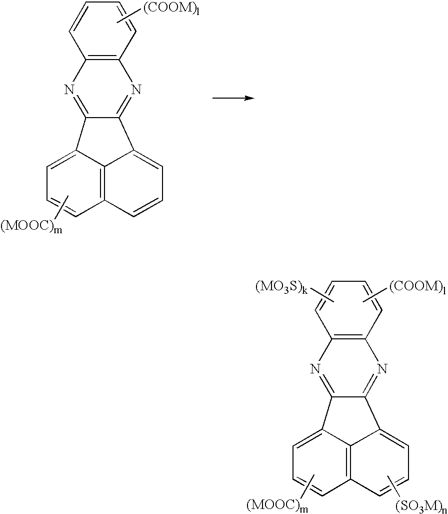

wherein: k, l are individually integers in the range of 0 to 4; m, n are individually integers in the range of 0 to 6; M is a counter ion.

These compounds can be also prepared by condensation of sulfo- and carboxy- derivatives of benzene-1,2-diamine and sulfo- and carboxyderivatives of acenaphthoquinone as follows:

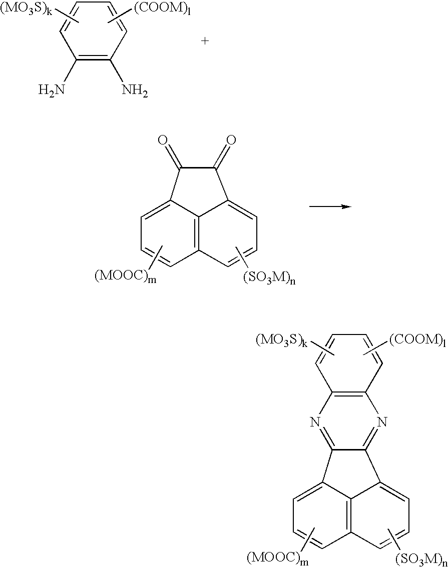

wherein: k, l are individually integers in the range of 0 to 4; m, n are individually integers in the range of 0 to 6; M is a counter ion.

The acenaphtho[1,2-b]quinoxaline sulfo- and carboxyderivatives of the present invention are capable of forming a LLC system that facilitates manufacturing of colorless anisotropic films represented by improved optical parameters.

The optical spectral characteristics and rheological properties of the sulfo- and carboxyderivative compounds indicate a strong tendency of the discotic dye molecules to aggregate, even in diluted aqueous solutions. These aggregates form LLC meso-phases in more-concentrated solutions. The supramolecular aggregates have a columnar structure, which is specific for flat elliptical shaped molecules grouped in a “face-to-face” fashion. The hydrophobic molecular planar cores of the aromatic conjugated bond system are stacked on each other inside of the aggregate, and the hydrophilic peripheral sulfonic carboxyl groups are exposed to water. Water provides a medium for electrostatic interaction and mutual alignment of supramolecules within resulting liquid crystal structure. The structure of the supramolecular aggregates creates a basis for multiple co-existing phases that may be viewed as a suspension of one phase in the other. Depending on the concentration and temperature of the LLC, there are two major phases: the hexagonal or “M-phase”, and the nematic or “N-phase”, in which supramolecules are about parallel but are not ordered along their cross sections.

The LLC material in N-phase can be deposited on film substrate using a slot-die coater. Molecular alignment during the shear deposition results in formation of coating with a strong preferred orientation, which remains in solid state after the drying. The resulting solid coating has natural characteristics of negative A-plate with fast axis along to the coating c-direction and refractive index about 1.48-1.52 along to the c-axis.

High degree of anisotropy and low depolarization can be achieved with optimal parameters of the coater and coating process, which correspond to: a) shearing of the liquid crystal coating prior to and during the coating process to provide alignment; b) three dimensional laminar flow during the coating process to maintain a high order of alignment during the transfer to the substrate; c) control of the post-coating process to preserve a well ordered, solid film.

Negative A-plate with fast axis along to the coating roll direction is suitable for roll-to-roll lamination with conventional sheet polariser because such a polarizer has the c-axis along to the roll direction (direction of the stretching).

Another application of these new LLC materials is a negative C-plate, which can be produced by aligning the columnar supramolecular aggregates perpendicular to a hydrophobic film substrate.

### Experimental

A number of experiments were conducted according the method and system of the present invention. These experiments are intended for illustration purposes only, and are not intended to limit the scope of the present invention in any way.

### EXAMPLE 1

Acenaphtho[1,2-b]quinoxaline-9-carboxylic acid was synthesized by condensation of Acenaphthenequinone with 3,4-Diaminobenzoic acid.

Dimethylformamide (1 l) was added to mixture of purified Acenaphthenequinone (20 g) and 3,4-Diaminobenzoic acid (16.74 g). Reaction mass was agitated and stored for 21 hours at room temperature. Precipitate was filtered and washed with dimethyl formamide and water. Yield 28 g.

### EXAMPLE 2

5-Sulfoacenaphtho[1,2-b]quinoxaline-9-carboxylic acid and 2-sulfoacenaphtho[1,2-b]quinoxaline-9-carboxylic acid were synthesized by sulfonation of acenaphtho[1,2-b]quinoxaline-9-carboxylic acid.

Acenaphtho[1,2-b]quinoxaline-9-carboxylic acid (3 g) was charged into 30% oleum (15 ml). Reaction mass was agitated at ˜70° C. for 17.5 hours. Obtained solution was diluted with water (33 ml) at 40-50° C. slowly. Reaction mass was agitated for overnight. The precipitate was filtered and dissolved in water (2 l). The solution of 5-Sulfoacenaphtho[1,2-b]quinoxaline-9-carboxylic acid and 2-sulfoacenaphtho[1,2-b]quinoxaline-9-carboxylic acid was charged into a feed tank and pumped to a UF cell. Permeate line was directed into a drain line while the more concentrated solution (retentate) was returned through a heat exchanger to the feed tank. During the process water was added to the feed tank to maintain level of the solution constant. The temperature of the solution was kept not higher than 45° C. The process was performed until electroconductivity of the permeate becomes roughly 20 μSm/cm and then allow volume in the feed tank to halve (cut off water). Continue circulating of the solution. When the electroconductivity became constant the volume of the solution was reduced twice and the ultrafiltration was stopped. Yield 3 g.

### EXAMPLE 3

Mixture of 1,2-dioxo-1,2-dihydroacenaphthylene-4-sulfonic acid and 1,2-dioxo-1,2-dihydroacenaphthylene-5-sulfonic acid was prepared by sulfonation of acenaphthenequinone.

Acenaphthenequinone (50 g) was charged into 20% oleum (150 ml) and agitated for 12 hours at ˜25° C. Obtained solution was diluted with water (140 ml) at 40-50° C. slowly. Reaction mass was stored for overnight. The precipitate filtered. The filter cake was suspended in acetic acid (300 ml). The precipitate was filtered and dissolved in acetone (200 ml). Obtained solution was diluted with dichloromethane (700 ml). The precipitate was filtered and dried on air without heating. Yield 23.5 g.

### EXAMPLE 4

Mixture of 2-sulfoacenaphtho[1,2-b]quinoxaline-9-carboxylic acid, 3-sulfoacenaphtho[1,2-b]quinoxaline-9-carboxylic acid, 4-sulfoacenaphtho[1,2-b]quinoxaline-9-carboxylic acid and 5-sulfoacenaphtho[1,2-b]quinoxaline-9-carboxylic acid (in equal amounts) were prepared by condensation of 1,2-dioxo-1,2-dihydroacenaphthylene-4-sulfonic acid and 1,2-dioxo-1,2-dihydroacenaphthylene-5-sulfonic acid with 3,4-Diaminobenzoic acid.

Suspension of 3,4-Diaminobenzoic acid (1.5 g) in acetic acid (30 ml) was added into suspension of 1,2-dioxo-1,2-dihydroacenaphthylene-4-sulfonic acid and 1,2-dioxo-1,2-dihydroacenaphthylene-5-sulfonic acid (2.6 g) in acetic acid (100 ml). Obtained reaction mass was agitated for 12 hours. Precipitate was filtered. Filter cake was dissolved in water (300 ml). The solution was filtered through fiber glass filter and diluted with concentrated hydrochloric acid (300 ml). The precipitate was filtered and dissolved in water (1 l). The solution of 2-sulfoacenaphtho[1,2-b]quinoxaline-9-carboxylic acid, 3-sulfoacenaphtho[1,2-b]quinoxaline-9-carboxylic acid, 4-sulfoacenaphtho[1,2-b]quinoxaline-9-carboxylic acid and 5-sulfoacenaphtho[1,2-b]quinoxaline-9-carboxylic acid was charged into a feed tank and pumped to a UF cell. Permeate line was directed into a drain line while the more concentrated solution (retentate) was returned through a heat exchanger to the feed tank. During the process water was added to the feed tank to maintain level of the solution constant. The temperature of the solution was kept not higher than 45° C. The process was performed until electroconductivity of the permeate becomes roughly 20 μSm/cm and then allow volume in the feed tank to halve (cut off water). Continue circulating of the solution. When the electroconductivity became constant the volume of the solution was reduced twice and the ultrafiltration was stopped. Yield 1 g.

### EXAMPLE 5

Water was added into the synthetic product obtained in Example 2 to prepare an aqueous solution to thereby obtain a lyotropic liquid crystal aqueous solution with a solid matter concentration of 24 wt %.

On the other hand, a triacetyl cellulose film showing almost no front retardation with a thickness retardation of almost 20 nm (with a thickness of 50 μm, manufactured Fuji Photo Film Co., Ltd. with a trade name of ZRF80S) was prepared.

The lyotropic liquid crystal aqueous solution was coated on one surface of the film so as to be a thickness of almost 800 nm (0.8 μm) after drying with a blade coater giving a shear for imparting orientation and thereafter, the wet coat was dried at 40° C. to obtain a light transmissive resin film.

The resin film was immersed in a 15% aqueous solution of barium chloride and thereafter the resin film was washed with water, dried under the following wind and subjected to a water-insoluble treatment to thereby obtain an optical film.

The front retardation value of the optical film was measured with a retardation measuring instrument (manufactured by Oji Sceientific Instruments with a trade name of KOBLA-31 PRW) to obtain almost 200 nm, which is a film having an optical anisotropy and can be used as a negative A plate.

### EXAMPLE 6

A polarizing plate was obtained according to an ordinary method. That is, a polyvinyl alcohol film was dyed in an aqueous solution containing iodine and crosslinked in a water bath containing boric acid and the like and thereafter, the polarizing plate was uniaxially stretched sixfold by having passed through between rolls different in speed from each other to thereby obtain a polarizer.

A triacetyl cellulose film (with a thickness of 80 μm, manufactured Fuji Photo Film Co., Ltd. with a trade name of T-50SH) was adhered to one surface of the polarizer with a polyvinyl alcohol based adhesive.

Then, the optically anisotropic film obtained in Example 5 was likewise adhered to the other surface of the polarizer with the polyvinyl alcohol based adhesive to thereby obtain a laminated optically anisotropic film.

What is claimed is:

1. An acenaphtho[1,2-b]quinoxaline sulfo- and carboxy-derivative represented by a structure formula selected from the group consisting of structures I: 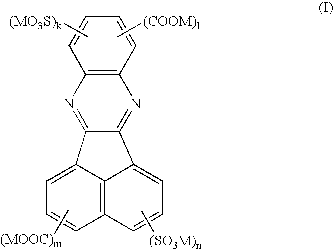
wherein: k, l are individually integers in the range of 0 to 4; m, n are individually integers in the range of 0 to 6; M is a counter ion.

2. The acenaphtho[1,2-b]quinoxaline sulfo- and carboxy-derivative according to claim 1, wherein said derivative is capable of forming a stable lyotropic liquid crystal system.

3. The acenaphtho[1,2-b]quinoxaline sulfo- and carboxy-derivative according to claim 1, wherein said derivative is capable of forming optically isotropic or anisotropic film.

4. A lyotropic liquid crystal system comprising at least one acenaphtho[1,2-b]quinoxaline sulfo- and carboxy-derivative represented by a structure formula selected from the group consisting of structures I: 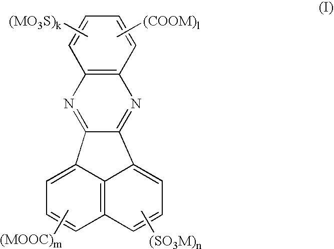
wherein: k, l are individually integers in the range of 0 to 4; m, n are individually integers in the range of 0 to 6; M is a counter ion.

5. The lyotropic liquid crystal system according to claim 4 further comprising a mixture of water and an organic solvent miscible with water.

6. The lyotropic liquid crystal system according to claim 5 further comprising up to approximately 30% by mass of surfactants.

7. The lyotropic liquid crystal system of according to claim 5 further comprising up to approximately 30% by mass of plasticizers.

8. The lyotropic liquid crystal system according to claim 4 further comprising at least one water-soluble organic compound capable of forming a common lyotropic liquid crystal system with at least one acenaphtho[1,2-b]quinoxaline sulfo- and carboxy-derivative of structure of I.

9. An optically anisotropic film comprising at least one acenaphtho[1,2-b]quinoxaline sulfo- and carboxy-derivative represented by a structure formula selected from the group consisting of structures I: 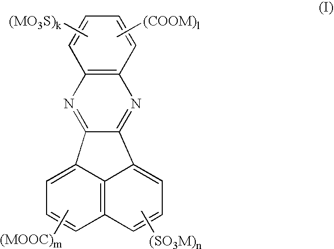
wherein: k, l are individually integers in the range of 0 to 4; m, n are individually integers in the range of 0 to 6; M is a counter ion.

10. A method of manufacturing an optically anisotropic film, comprising the step of:
depositing a lyotropic liquid crystal system according to claim 4 onto a substrate;
applying an orienting force; and
drying.

11. The method of manufacturing the optically anisotropic film according to claim 10, wherein said film is formed between at least two substrates with oriented or rubbed surface from the lyotropic liquid crystal system according to claim 4.

12. The optically anisotropic film of claim 9, wherein said optically anisotropic film is at least partially crystalline.

13. The optically anisotropic film of claim 9, wherein the interplane spacing in a crystal is in the range of approximately 3.1 Å to 3.7 Å along one of the optical axes.

14. The optically anisotropic film of claim 9, wherein said optically anisotropic film is a birefringent film.

15. The optically anisotropic film of claim 9, wherein said optically anisotropic film is a negative A-plate.

16. The optically anisotropic film of claim 9, wherein said optically anisotropic film is a negative C-plate.

17. The optically anisotropic film of claim 9, wherein said optically anisotropic film is a polarizing plate.

18. A laminated optical film in which at least one optically anisotropic film is laminated with an optical multilayer film having at least one polarizer, wherein
said optically anisotropic film comprises at least one acenaphtho[1,2-b]quinoxaline sulfo- and carboxy-derivative represented by a structure formula selected from the group consisting of structures I: 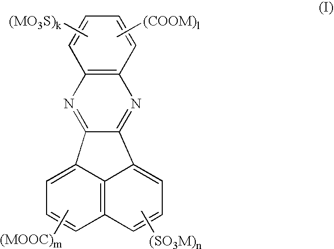
wherein: k, l are individually integers in the range of 0 to 4; m, n are individually integers in the range of 0 to 6; M is a counter ion.

19. The laminated optical film according to claim 18, wherein said optically anisotropic film is a negative A-plate.

20. The laminated optical film according to claim 18, wherein said optically anisotropic film is a negative C-plate.

21. A laminated optical film in which at least one optically anisotropic film is laminated with an optical multilayer film having at least one retarder, wherein said optically anisotropic film comprises at least one acenaphtho[1,2-b]quinoxaline sulfo- and carboxy-derivative represented by a structure formula selected from the group consisting of structures I: 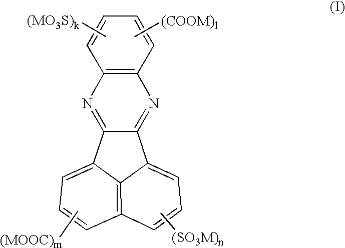
wherein: k, 1 are individually integers in the range of 0 to 4; m, n are individually integers in the range of 0 to 6; M is a counter ion.
